(19) United States
(12) Patent Application Publication
(10) Pub. No.: US 2021/0035664 A1
(43) Pub. Date: Feb. 4, 2021

(54) ELECTRONIC HEALTHCARE RECORD SYSTEM

(71) Applicant: Vericle Corporation, Clearwater, FL (US)

(72) Inventors: Yuval LIROV, Clearwater Beach, FL (US); Erez LIROV, Morganville, NJ (US)

(21) Appl. No.: 16/531,076

(22) Filed: Aug. 4, 2019

(51) Int. Cl. G16H 10/60; G06F 16/242; G16H 50/70; G06F 9/451

(52) U.S. Cl. CPC G16H 10/60; G06F 9/451; G16H 50/70; G06F 16/242

(57) ABSTRACT

An electronic healthcare record (EHCR) data processing method is provided herein the method comprising: running an EHCR program of code that comprises four separate but interactive layers of software code comprising—a view layer of software code (view layer), a template layer of software code (template layer), a platform layer of software code (platform layer), and a database layer of software code (database layer).

### CROSS REFERENCE TO RELATED APPLICATIONS

Related subject matter is disclosed in several co-pending U.S. Design and Utility Non-provisional patent applications, with the following Serial No's and filing dates, the entire contents of each of which are expressly incorporated herein by reference: 29/521,340, filed Mar. 23, 2015; Ser. No. 29/525,160 filed Apr. 27, 2015; Ser. No. 29/600,900, filed Apr. 17, 2017; Ser. No. 14/665,502, filed Mar. 23, 2015; Ser. No. 29/524,080, filed Apr. 16, 2015, Ser. No. 16/248,725, filed Jan. 15, 2019; and Ser. No. 29/668,522, filed Oct. 31, 2018.

### TECHNICAL FIELD

The embodiments described herein relate generally to electronic data management software programs, and more specifically to systems, methods, and modes for a healthcare record management, collection, and retention software program, and interactions between people and the healthcare records software program.

### BACKGROUND

There are several significant problems with currently available medical/healthcare electronic record keeping programs and systems. Even though many of these currently available healthcare record keeping programs have many and useful features, such features are all but useless if the programs themselves are inherently non-intuitive to use, difficult to maintain, inefficient, and/or any combination of these problems, or more.

Consequently, the rate of acceptance of electronic medical record (EMR) programs/systems remains low; in addition to the issues discussed above, other problems include impeding visit documentation processes, and increase clinical decision error counts, instead of reducing them. Further, EMR systems lack key functionality, remain difficult to use, are slow to respond, and difficult to customize.

In response to the problems discussed above, manufacturers of currently available EMR systems have created very complicated coding, in which all functions and components were tightly coupled together. This complicated coding created its own set of problems. Consequently, manufacturers then simplified the coding, reducing functionality. Further, performance was comprised, the programs lost ease of use, and created long customization delays. In addition to the long customization delays, there were also long development cycles. Once such systems were installed, updates became risky to implement because a change in one part of the system would often disrupt other parts. At some point manufacturers decided to improve the EMR systems by increasing the number of specialties that could use the EMR system, and code complexity rose geometrically, repeating the cycle.

Thus, there is a need for systems, methods, and modes for an EMR system that improves functionality and interactions between people and the EMR, remains relatively easy to update, provides a better match between healthcare workflow and the EMR system, improves clinician teamwork, accelerates the documentation process, reduces medical billing mistakes, and improves documentation and billing compliance.

### SUMMARY OF THE INVENTION

An object of the embodiments is to substantially solve at least the problems and/or disadvantages discussed above, and to provide at least one or more of the advantages described below.

It is therefore a general aspect of the embodiments to provide systems, methods, and modes for an EMR system that improves functionality and interactions between people and the EMR, remains relatively easy to update, provides a better match between healthcare workflow and the EMR system, improves clinician teamwork, accelerates the documentation process, reduces medical billing mistakes, and improves documentation and billing compliance that will obviate or minimize problems of the type previously described.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Further features and advantages of the aspects of the embodiments, as well as the structure and operation of the various embodiments, are described in detail below with reference to the accompanying drawings. It is noted that the aspects of the embodiments are not limited to the specific embodiments described herein. Such embodiments are presented herein for illustrative purposes only. Additional embodiments will be apparent to persons skilled in the relevant art(s) based on the teachings contained herein.

According to a first aspect of the embodiments, and electronic healthcare record (EHCR) data processing method is provided comprising: running an EHCR program of code that comprises four separate but interactive layers of software code comprising—a view layer of software code (view layer), a template layer of software code (template layer), a platform layer of software code (platform layer), and a database layer of software code (database layer).

According to the first aspect of the embodiments, the view layer is adapted to render template code to produce one or more interactive graphical user interfaces for entering and displaying data.

According to the first aspect of the embodiments, the template layer is adapted to store data entered as notes during visits by patients to a healthcare facility.

According to the first aspect of the embodiments, the template layer is further adapted to render the notes data in narrative form.

According to the first aspect of the embodiments, the template layer is further adapted to obtain medical information from the database layer and generate treatment recommendations based on the obtained medical information.

According to the first aspect of the embodiments, the template layer is further adapted to obtain data from both the platform layer and database layer.

According to the first aspect of the embodiments, the platform layer of code is adapted to respond to data requests from the template layer.

According to the first aspect of the embodiments, the platform layer, in response to data requests from the template layer, generates database search queries and retrieves the requested data from the appropriate database located in the database layer.

According to the first aspect of the embodiments, the database layer is adapted to store data in one or more separate database storage locations.

According to the first aspect of the embodiments, the database layer is further adapted to store graphical user interface (GUI) code.

According to the first aspect of the embodiments, the method further comprises: displaying the GUI code as a template GUI in a view layer of code.

According to a second aspect of the embodiments, a method for operating an electronic healthcare record system is provided, the method comprising: operating software code that is organized into four separate layers, and wherein the software code comprises—a view layer of software code (view layer), a template layer of software code (template layer), a platform layer of software code (platform layer), and a database layer of software code (database layer); rendering the template layer to display one or more interactive graphical user interfaces (GUIs) for use in entering, storing, retrieving, and processing electronic healthcare data, wherein the rendering occurs in the view layer; maintaining data entered as notes documenting a visit by a patient, wherein the notes are maintained in the template layer; generating a narrative for each set of notes, wherein the narrative is generated by software code maintained in the template layer; requesting additional data through use of the one or more interactive GUIs; obtaining requested data through the platform layer; and storing requested data, notes data, and narratives in the database layer.

According to a third aspect of the embodiments an electronic healthcare record (EHCR) data processing system is provided comprising: at least one electronic device, wherein the electronic device comprises—at least one processor adapted to execute software code, at least one display adapted to view results of executed software code, and at least one memory storage adapted to store the executable software code; and executable software code, wherein the executable software code comprises—a view layer of software code (view layer), a template layer of software code (template layer), a platform layer of software code (platform layer), and a database layer of software code (database layer).

According to the third aspect of the embodiments, the view layer is adapted to render template code to produce one or more interactive graphical user interfaces for entering and displaying data.

According to the third aspect of the embodiments, the template layer is adapted to store data entered as notes during visits by patients to a healthcare facility.

According to the third aspect of the embodiments, the template layer is further adapted to render the notes data in narrative form.

According to the third aspect of the embodiments, the template layer is further adapted to obtain medical information from the database layer and generate treatment recommendations based on the obtained medical information.

According to the third aspect of the embodiments, the template layer is further adapted to obtain data from both the platform layer and database layer.

According to the third aspect of the embodiments, the platform layer of code is adapted to respond to data requests from the template layer.

According to the third aspect of the embodiments, the platform layer, in response to data requests from the template layer, generates database search queries and retrieves the requested data from the appropriate database located in the database layer.

According to the third aspect of the embodiments, the database layer is adapted to store data in one or more separate database storage locations.

According to the third aspect of the embodiments, the database layer is further adapted to store graphical user interface (GUI) code.

According to the third aspect of the embodiments, the system further comprises: displaying the GUI code as a template GUI in a view layer of code.

### BRIEF DESCRIPTION OF THE DRAWINGS

The above and other objects and features of the embodiments will become apparent and more readily appreciated from the following description of the embodiments with reference to the following figures, wherein like reference numerals refer to like parts throughout the various figures unless otherwise specified, and wherein:

FIG. 1 illustrates a block diagram of a medical enterprise location within with an electronic healthcare record system can be utilized according to aspects of the embodiments.

FIG. 2 illustrates a block diagram of a functional relationship between major software components of an electronic healthcare record system software program according to aspects of the embodiments.

FIG. 3 illustrates a detailed block diagram view of the functional relationship between major software components of an electronic healthcare record system software program according to aspects of the embodiments.

FIG. 4 illustrates a flow diagram of a method of operation of the electronic healthcare record system software program according to aspects of the embodiments.

FIG. 5 illustrates a flow diagram of receiving and inputting electronic healthcare data records within the medical enterprise location of FIG. 1 using the electronic healthcare record system according to aspects of the embodiments.

FIG. 6 illustrates an example of one of a plurality of graphical use interfaces illustrating input of notes-data into the electronic healthcare record system and their subsequent transformation into a narrative form that can be stored and disseminated according to aspects of the embodiments.

FIG. 7 illustrates a data flow diagram of note data from a plurality of specialists into the electronic healthcare record system and their transformation into narratives including shared content according to aspects of the embodiments.

FIGS. 8-10 illustrate a series of graphical user interfaces/screenshots that illustrate exemplary but non-limiting embodiments of electronic interfaces with the electronic healthcare record system software program according to aspects of the embodiments.

FIG. 11 illustrates a block diagram of a computer/server/tablet/personal electronic device that can be used with the electronic healthcare record system software program according to aspects of the embodiments.

FIG. 12 illustrates a computer network system within which the electronic healthcare record system software program can be utilized according to aspects of the embodiments.

### DETAILED DESCRIPTION OF THE INVENTION

This Application incorporates by reference the Appendix filed herewith.

The embodiments are described more fully hereinafter with reference to the accompanying drawings, in which embodiments of the inventive concept are shown. In the drawings, the size and relative sizes of layers and regions may be exaggerated for clarity. Like numbers refer to like elements throughout. The embodiments may, however, be embodied in many different forms and should not be construed as limited to the embodiments set forth herein. Rather, these embodiments are provided so that this disclosure will be thorough and complete, and will fully convey the scope of the concept to those skilled in the art. The scope of the embodiments is therefore defined by the appended claims. The following embodiments are discussed, for simplicity, with regard to the terminology and structure of a personal electronic device, typically a tablet (such as an iPad, sold by Apple, Inc.) and server computer (server) and/or personal computer (PC) and a local, wide, or global area network system, as used in one or more medical offices. However, the embodiments to be discussed next are not limited to these systems but may be applied to other types of medical enterprise locations, such as hospitals, emergency care facilities, health care centers, and the like, whether comprising one or multiple, distributed offices.

The following is a list of acronyms used in alphabetical order:
3G Third Generation
4G Fourth Generation
5G Fifth Generation
App Executable Software Programming Code/Application
BT BlueTooth
CD Compact Disk
DC Doctor Of Chiropractor
DVD Digital Video Disk
EE Electrably Erasable
EHCR Electronic Healthcare Record
HDD Hard Disk Drive
HDMI High Definition Multimedia Interface
ISP Internet Service Provider
LTE Long Term Evolution
MD Medical Doctor
NFC Near Field Communications
PC Personal Computer
PED Personal Electronic Device
POTS Plain Old Telephone Service
PROM Programmable Read Only Memory
PT Physical Therapist
RAM Random Access Memory
ROM Read-Only Memory
RW Read/Write
USB Universal Serial Bus (USB) Port
UV Ultra-Violet
VGA Video Graphics Array

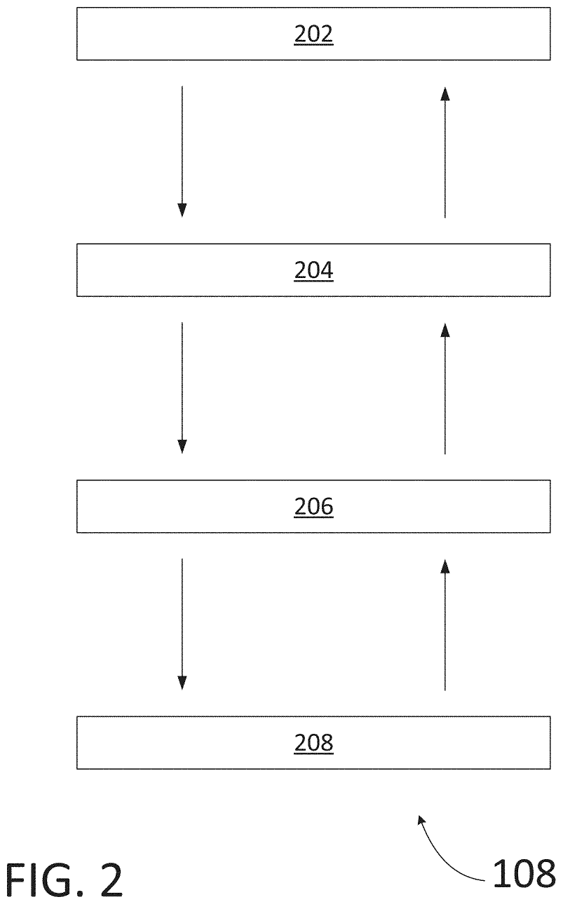

The following is a list of the elements of the Figures in numerical order:
100 Electronic Healthcare Record System
102 Personal Computer/Tablet/Laptop/Personal Electronic Device (PC; PED)
104 Processor
106 Memory
108 Display (Touch Screen)
110 Electronic Healthcare Record Software Program (EHCR Program)
112 Data/Command Bus
114 Server
116 Database Storage Device
118 Patient
120 Physician
122 Healthcare Technician
124 Nurse
126 Administrator of Electronic Healthcare Record System
128 Wired/Wireless Network Interconnection
202 View Layer
204 Template Layer
206 Platform Layer
208 Database Layer
400 Electronic Healthcare Record System Method of Operation (Between Functional Layers of FIG. 2)
402-410 Method Steps of Method 400
500 Block Diagram Of Process for Receiving and Inputting Electronic Healthcare Data Records
502 Intake Form/Patient Portal Block
504 Intake Form/Check-in Kiosk Block
506 Initial Exam/Eval Block
508 Daily Notes Block
510 Re-Exam Block
512 Discharge Block
600 Graphical User Interface for Inputting Medical/Health Data
602 Data Entry Window for GUI 600
604 Automatically Formed and Formatted Narrative Display for GUI 600 Based on Data Entered in Window 602
700 Data Flow Diagram
702 Doctor of Chiropractor
704 Medical Doctor
706 Physical Therapist
708 Data Retention Unit
710 DC Note(s)
712 MD Note(s)
714 PT Note(s)
716 DC Narrative(s)
718 MD Narrative(s)
720 PT Narrative(s)
800 Template Selection GUI
802 Interface Window
804 Search Button
806 Patient List Window
808 Documentation Workspace Selection Window
810 Chiropractic Documentation Workspace
812 Documentation Preview Window
814 External Programs Column
902 Chiropractor Interactive GUI
904 Subjective Interactive Data Entry Tab
906 Subjective Interactive Data Entry Window
908 Collapse Button
1000 Physical Therapy GUI
1002 Daily Living Record Field
1004 Add Button
1006 Macro Field
1008 Activity Button
1110 Universal Serial Bus (USB) Port
1111 Ethernet Port
1112 Compact Disk (CD)/Digital Video Disk (DVD) Read/Write (RW) (CD/DVD/RW) Drive
1114 Floppy Diskette Drive
1116 Hard Disk Drive (HDD)
1118 Read-Only Memory (ROM)
1120 Random Access Memory (RAM)
1122 Video Graphics Array (VGA) Port or High Definition Multimedia Interface (HDMI)
1124 External Memory Storage Device
1126 External Display/Touch-Screen
1128 Keyboard
1130 Mouse
1132 Processor Board/PC Internal Memory (Internal Memory)
1134 Flash Drive Memory
1136 CD/DVD Diskettes
1138 Floppy Diskettes
1142 Wi-Fi Transceiver
1144 BlueTooth (BT) Transceiver
1146 Near Field Communications (NFC) Transceiver
1148 Third Generation (3G), Fourth Generation (4G), Long Term Evolution (LTE), 5G (Fifth Generation) (3G/4G/LTE/5G) Transceiver
1150 Communications Satellite/Global Positioning System (Satellite) Transceiver Device
1152 Antenna
1154 Internet
1156 Universal Serial Bus (USB) Cable
1158 Ethernet Cable (CATS)
1160 Scanner/Printer/Fax Machine
1200 Network System
1206 Internet Service Provider (ISP)
1208 Modulator/Demodulator (Modem)
1210 Wireless Router
1212. Plain Old Telephone Service (POTS) Provider
1214 Cellular Service Provider
1218 Communication Satellites
1220 Cellular Telecommunications Service Tower (Cell Tower)
1224 GPS Station
1226 Satellite Communication Systems Control Station
1228 Global Positioning System (GPS) Satellite
1230 Server

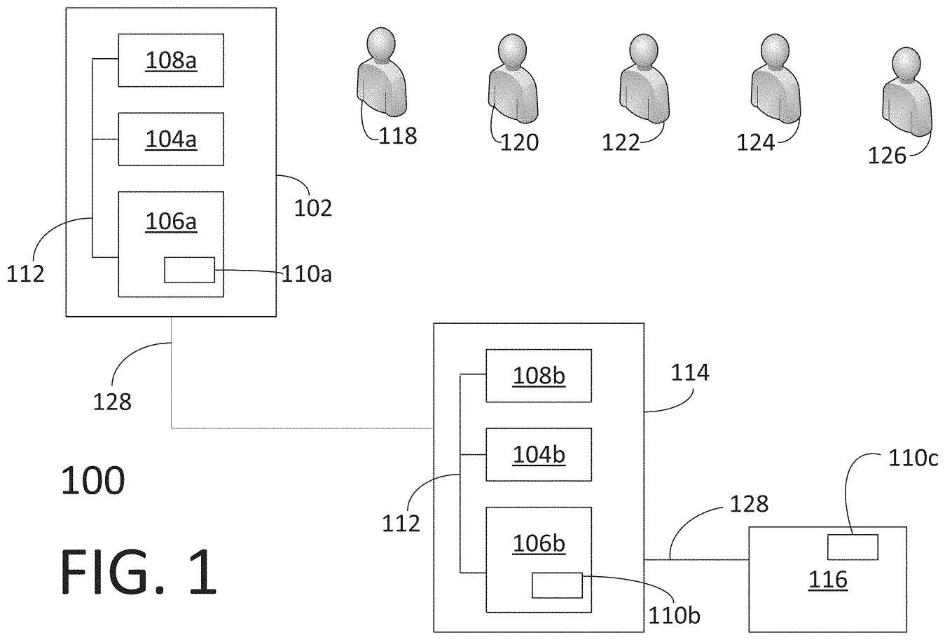

FIG. 1 illustrates a block diagram of a medical enterprise location within with an electronic healthcare record (EHCR) system 100 can be utilized according to aspects of the embodiments. EHCR system 100 comprises one or more of one or more computers 102, one or more servers 114, and database memory storage device (database memory) 114. In addition, EHCR system 100 further comprises EHCR program 110. Users of EHCR system 100 and EHCR program can include one or more of patients 118, physicians 120, healthcare technicians/physical therapists 122, nurses 124, and administrators 126

In FIG. 1, the device labeled 102 is shown as a tablet, or PED 102; such PED 102 can be one or more of a personal computer (PC), laptop, tablet (such as an iPad, sold by Microsoft, Inc., or other similar devices), and a kiosk device, all of which are capable of storing and running one or more programs or applications, have processors 104, displays 108 (in this case, interactive touch screens, though such need not be the case), memory 106, storing all or part of EHCR program 110. In addition, there is also data/command buses 112, and network interface hardware/software that provides for electronic communications via wired/wireless communications interface 128, according to aspects of the embodiments. Such components and circuitry of modern electronic processing devices are well known to those of skill in the art, with the except of EHCR program 110, and thus, in fulfillment of the dual purposes of clarity and brevity, a detailed discussion of the same has been omitted from herein.

In the medical enterprise location of FIG. 1, however, for the non-limiting purposes of this discussion, EHCR system 100 is shown to comprise one or more PEDs 102, server 114, and a separate database memory storage device 116, all of which are connected by one or more of wired/wireless means typically, though not necessarily, and therefore not to be taken in a limiting manner, an ethernet network interface and cable. One or more of the PEDs 102, server 114, and database 116 can use wireless means as well, including, but not limited to near field communications (NFC) devices, WiFi, BlueTooth, among others, and the like.

Further, as shown in FIG. 1, EHCR program 110 is shown as comprising three major parts: EHCR program 110a stored in memory 106a of PED 102, EHCR program 110b stored in memory 106b of server 114, and EHCR program 110c stored in database 116, according to aspects of the embodiments. While this is one possible configuration of EHCR program 110, it is also possible that EHCR program 110 can be stored all on one device, such as a PED 102, or server/pc 114. Nonetheless, in fulfillment of the dual purposes of clarity and brevity, discussion shall be made of the configuration as shown in FIG. 1, described above. Such allocation of the components of EHCR program 110 is not to be taken in a limiting sense.

In the configuration of devices and EHCR program 110 as shown in FIG. 1, it is likely that one or more of patient 118, doctor 120, technician 122, and nurse 124 could accesses EHCR program 110 via PED 102 and EHCR program 110a, and administrator 126 can access EHCR program 110 via server/PC 14, and EHCR program 110b,c. As those of skill in the art can appreciate, the manner of interfacing with EHCR program 110 by the aforementioned individuals is relatively known to those of skill in the art; patients 118 enter data or check in to their visit via PED 102 and EHCR program 110a. Nurses 124 can record vital signs and other health/personal information directly on the same PED 102, using EHCR program 110a, and if a physician's review/care/visit is necessary, she too can access the same set of records and information via PED 102 and EHCR program 110a. Once the visit is complete, technician 124 can use either of PED 102 or server 114 and completing billing and/or follow-up visit matters, and administrator 126 can implement updates and/or perform maintenance to EHCR program 110 as needed, among many other tasks.

FIG. 2 illustrates a block diagram of a functional relationship between major software components of electronic healthcare record system software program 110 according to aspects of the embodiments. According to aspects of the embodiments, ECHR program 110 comprises four separate layers of code that are functionally different from each other; this novel, non-obvious, and improved software program is a technological improvement over previous known electronic health care record programs. It provides a practical application of a software program by applying novel and unobvious programming solutions to real, physical devices and business operations that ultimately facilitates the recordation and processing of healthcare data that obviates the problems of known systems and programs. According to aspects of the embodiments, EHCR program 110 matches healthcare practice workflow, improves inter-clinician teamwork, accelerates the documentation process, reduces billing mistakes, and improves documentation and billing compliance.

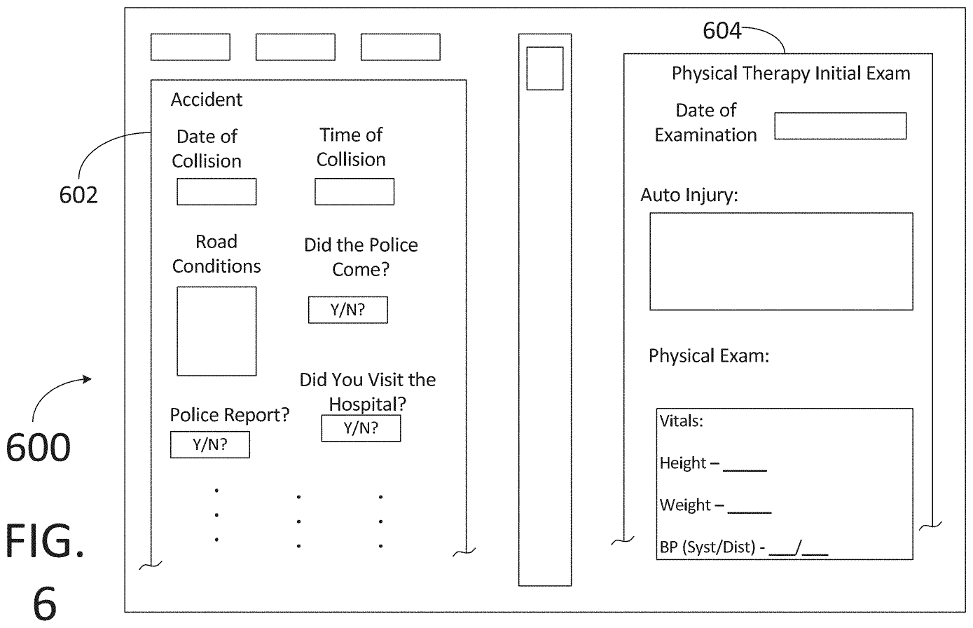

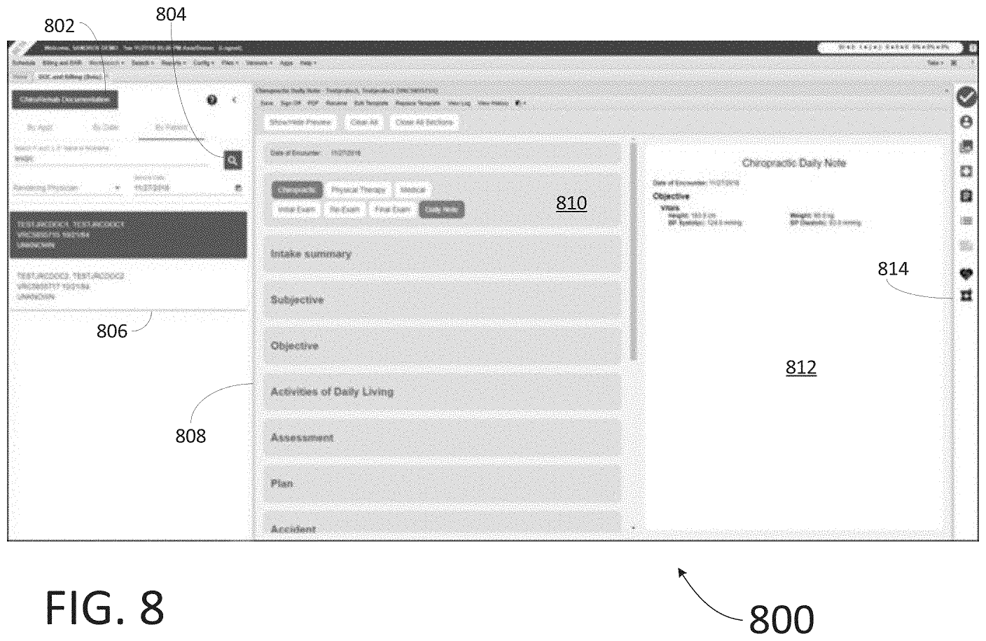

Referring now to FIG. 2, view layer 202, according to aspects of the embodiments, includes software code that renders the computer screen for the best user interaction experience to match the workflow from patient scheduling to patient check in, to documentation, to billing. View layer 202 receives data directly input into interactive windows in graphical user interfaces it generates, and retrieves templates to place such data, as well data that has been previously recorded, from template layer 204. That is, the code of view layer obtains templates in the form of code/sub-routines, and the like, and renders it into templates that are presented are GUIs; FIGS. 6 and 8 represent such GUIs; FIG. 6 is discussed in greater detail below. FIG. 8 illustrates template selection GUI 800 that can be a main template selection GUI for use with EHCR program 110 according to aspects of the embodiments.

Upon opening EHCR program 110, and entering username and password information, a user (120. 122, 124, 126, and 128), would encounter template selection GUI 800. At interface window 802, the user can select from the pull down window any number of templates, or it can enter search criteria, and click on the search button 804 to find a desired template. These and other template features are well known to those of skill in the art, and therefore, in fulfillment of the dual purposes of clarity and brevity, will not be discussed in greater depth or detail, but are shown to illustrate the interaction between layers 202-208 according to aspects of the embodiments.

A patient list can also be displayed in window 806; if the patient is already entered into the system, their most recent set of notes can be selected and displayed. In documentation workspace selection window 808, a particular document workspace template can be selected; in FIG. 8 the chiropractic documentation workspace 810 has been selected, and its corresponding documentation preview window 812 is shown. To the right of window 812 is shown external programs column 814, from which a user can select an external program to use in conjunction with documentation preview window 812 or other aspects of EHCR program 110 according to aspects of the embodiments.

The next layer is template layer 204. Template layer 204, as its name implies, stores the code that embodies all of the templates, both interactive and non-interactive, that a user will encounter in using EHCR program 110 according to aspects of the embodiments. Template layer 204 also maintains note documentation intelligence, such as the visit kind for different specialties. That is, as those of skill in the art can appreciate, different medical healthcare specialists can have different requirements in the types of information that they want to note or document; as such, there can be different sets of templates for the different specialists in EHCR program 110 according to aspects of the embodiments. Furthermore, template layer 204 maintains the code for automated data driven narrative generation and context awareness that automatically determines needed tests dependent on earlier test results. Template layer 204 creates data requests for platform layer 208, described below.

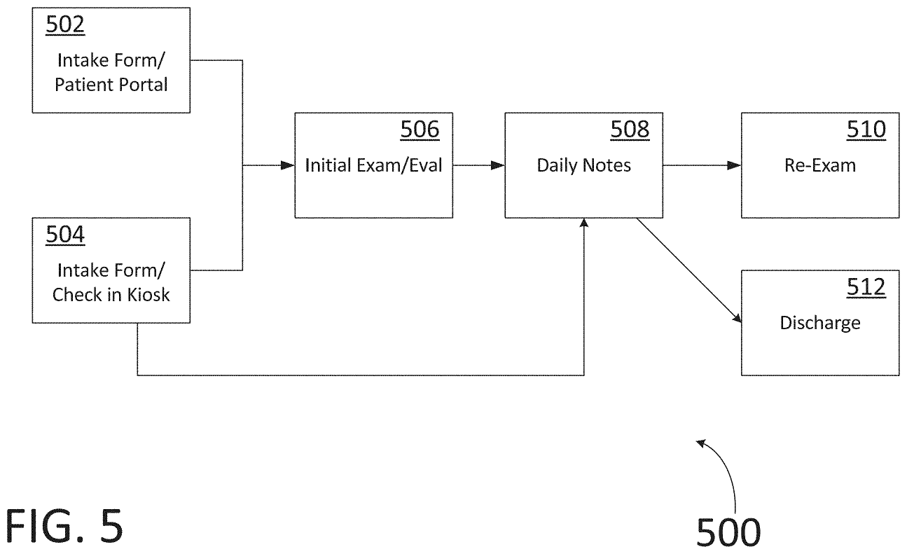

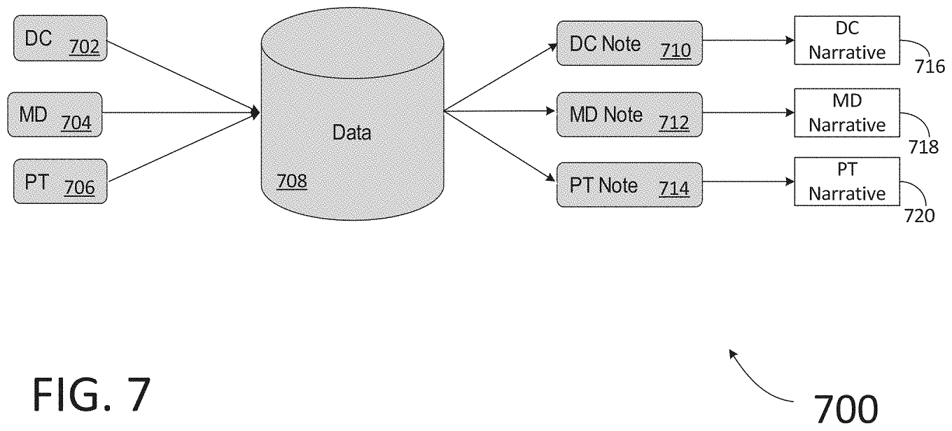

Data Driven Narrative Generation: Attention is directed to FIGS. 5-7. FIG. 5 illustrates flow diagram 500 of receiving and inputting electronic healthcare data records within the medical enterprise location of FIG. 1 using electronic healthcare record system 100 according to aspects of the embodiments, FIG. 6 illustrates an example of one of a plurality of graphical use interfaces 600 illustrating input of notes-data into electronic healthcare record system 100 and their subsequent transformation into a narrative form that can be stored and disseminated according to aspects of the embodiments, and FIG. 7 illustrates data flow diagram 700 of note data from a plurality of specialists into the electronic healthcare record system and their transformation into narratives including shared content according to aspects of the embodiments. In data flow diagram 500, it can be seen that in blocks 502 and 504 there are two main ways for a patient's data to be entered into ECHR program 110 according to aspects of the embodiments. In block 502, which represents one or more templates that patient 118 or other users 120, 122, 124, 126 can access and enter initial patient information. In data block 504 such patient data is entered via a kiosk by patient 118, or self-use interactive device (which can be in the form of PED 102 or some other electronic interactive device). In either case, the patient data, once entered, becomes available for review by nurse 124, technician 122, or physician 120 during an initial examination/evaluation, as shown in block 506.

Any data entered into blocks 502, 504, 506 show up in the daily notes, shown in block 508 and described briefly above in regard to FIG. 8. Daily notes are the data and information that nurse 124, physician 120, and/or technician 122 enter into a respective template—such daily notes are then converted to a narrative, which is an automatically data populated document that provides in summary prose form the data from the daily notes and other data sources. The narrative is shown in FIG. 6; once the daily notes are completed, then a re-exam can be ordered (block 510; and the process begins anew) or the patient discharged and billing can occur (block 512).

FIG. 6 illustrates an example of one of a plurality of graphical use interfaces 600 illustrating input of notes-data into electronic healthcare record system 100 and their subsequent transformation into a narrative form that can be stored and disseminated according to aspects of the embodiments. In GUI 600 there are two main fields: data entry window 602 (the daily notes referenced above) and automatically formed and formatted narrative display for GUI 600 based on data entered in data entry window 602 (as well as data previously entered, as described in regard to FIG. 5). A user can enter data in the data entry fields shown in FIG. 6 in window 602 and template layer 204 converts the data into a narrative that is much more easily read and comprehensive according to aspects of the embodiments. FIG. 7 illustrates data flow diagram 700 of note data from a plurality of specialists into the electronic healthcare record system and their transformation into narratives including shared content according to aspects of the embodiments. As can be seen in FIG. 7, data flows from any number of sources into ECHR program 110 and stored in data storage 708, from which notes 710, 712, and 714 are generated and then automatically converted into narratives 716, 718, 720 according to aspects of the embodiments.

Context Awareness: According to aspects of the embodiments, context awareness is a feature built into template layer 204 of ECHR program 110 that may or may not be readily apparent to users of the program. That is, context awareness is code included in template layer 204 that generates recommendations that can include one or more of additional tests, treatment, medication, physical therapy, lifestyle recommendations (exercise, sleep, rest, eating and drinking habits, among others), holistic therapies, and counseling, among other types of preventative and remedial measure based on reviews of previous test data results, as well as any other data that is associated with the patient. Such coding is a form of artificial intelligence, and additional recommendations can be modified based on results from previous recommendations and the effects they had on the patient. In addition to the above template layer 204 creates data requests for platform layer, according to aspects of the embodiments, and seeks to obtain almost instantaneous responses.

The next layer below template layer 204 is platform layer 206. The chief function of platform layer is to either provide data stored therein, or retrieve it from database layer 208. As its name implies, database layer is the data storage layer: within database layer there is such databases as the patient database, visit schedule database, providing clinician database, medical diagnosis database, medical procedure code database, among others. According to further aspects of the embodiments, database layer 208 also stores the code for the templates that can be accessed via template layer 204.

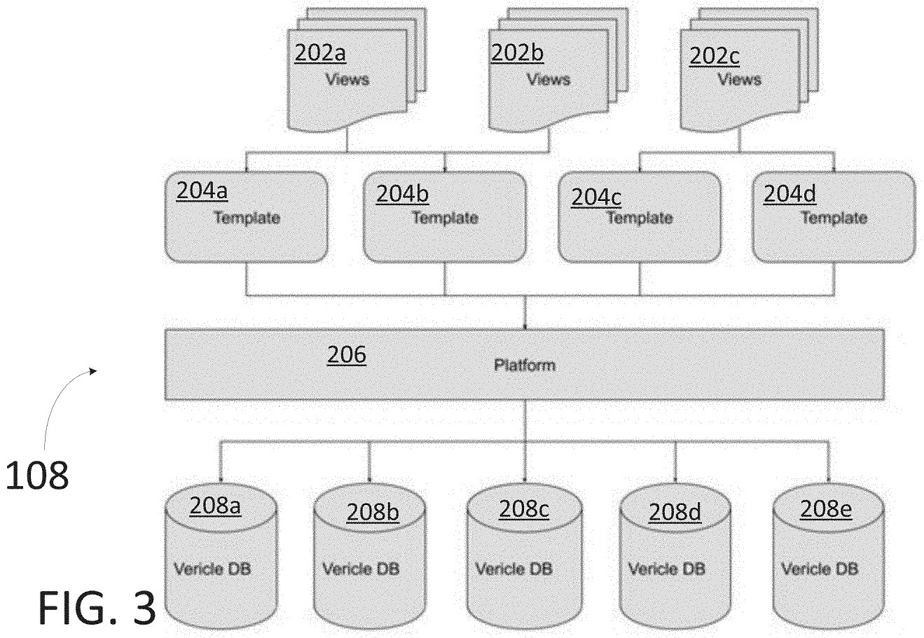

FIG. 3 illustrates a detailed block diagram view of the functional relationship between major software components of EHCR program 100 according to aspects of the embodiments. The block diagram of FIG. 3 shows in greater detail the relationships between the different functional layers 202, 204, 206 and 208 in EHCR program 100 according to aspects of the embodiments. That is, as opposed to one homogenous set of code for view layer 202, there are sets of code, 202a, 202b, 202c, and so on, that represent major sub-functional blocks within functional layer 202. For example view layer 202a could represent the set of code that is based on a first specialty, 202b for a second specialty, and so on, and 202n could be the set of code for view layer 202 that pertains to billing. Then, what follows is that below that are the different sets of template layer code 204 that is particular to the specialties above it; that is, by way of non-limiting example, if view layer 202a pertains to an ob-gyn physician, and view layer 202b pertains to pediatric physician(s) sharing the same office, then templates 204a can be specific to the practices of the ob-gyn physician(s), and templates 204b can be specific to the practices of the pediatrics physician(s). Views 202c can be the views that are needed by the office and billing manager; however, platform 206 provides all the code for the template layer 204, and of course databases 208a-e, which can be created separately to make data extraction more efficient, can still be shared as there are circumstances when billing, by way of non-limiting example, will need to access some visit information (e.g., if tests/supplies were used, and needed to be billed), according to aspects of the embodiments.

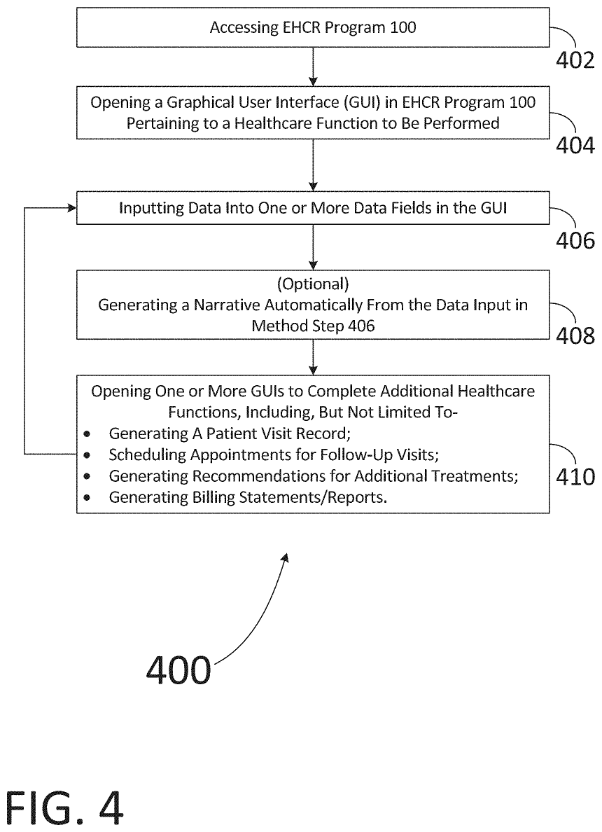

FIG. 4 illustrates a flow diagram of a method of operation of the electronic healthcare record system software program (method) 400 according to aspects of the embodiments. Method 400 begins with a user (120, 122, 124, or 126) accessing EHCR program 100 (typically, entering a username and password), and then opening a first graphical user interface (GUI) that pertains to a healthcare function to be performed; such healthcare functions can include initial patient data entry, a revisit, physical therapy, billing, among many others. Then, in method step 406, the user enters data into the opened GUI. An optional step that can occur after method step 406 is the automatic generation of a narrative based on data entered into one or more specific data fields upon which narratives can be generated in optional method step 408. That is, not all data entered will be used to generate a narrative. Narrative, according to aspects of the embodiments, are a summary, in prose form, of specific data for specific purposes in a predefined format. That is, the narrative is produced, as described above, by code that generates in paragraph form the data entered in one or more data entry windows/fields found in one or more GUIs. In essence, it makes the entered data more readable, according to aspects of the embodiments.

Following method step 408, in method step 410, the user can open and use additional GUIs that can be used to perform other healthcare related functions such as, but not limited to generating a patient visit record, scheduling appointments for follow-up visits, generating recommendations for additional treatments, and generating billing statements/reports, among many other healthcare functions.

As those of skill in the art can appreciate, both the discussion and illustration of method 400 for using EHCR program 100 as presented herein is very much simplified for the purposes of this discussion. Many other ancillary operations/steps can occur; for example, as shown in FIG. 8, discussed above, documentation preview window 812 is shown. To the right of window 812 is shown external programs column 814, from which a user can select an external program to use in conjunction with documentation preview window 812 or other aspects of EHCR program 110 according to aspects of the embodiments.

According to further aspects of the embodiments, as the user operates EHCR program 100, the different layers 202-208 are used, in the manner as described above. That is, upon opening EHCR program 100, view layer 202 retrieves code from database layer 208 for the appropriate template that will create the GUI that the user desires to see; that command or request for a specific set of code is passed from view layer 202, to template layer 204, to platform layer 206, and then to database layer 208, and then back up again to produce the GUI. According to aspects of the embodiments, such delineation of functionality creates synergistic opportunities in regard to overall perform of EHCR program 100. Designs that lack such separation of responsibilities between view layer 202, template layer 204, platform layer 206, and database layer 208 result in very tight coupling of components in the overall system. As those of skill in the art can appreciate, such tight coupling creates long development cycles with very risky updates where a change in one part of the system could very easily disrupt the behavior of all other parts. Furthermore, as more specialties and visit types are added, the complexity increases, and therefore the risk grows geometrically. According to aspects of the embodiments, updates can be made to individual layers 202-208 of EHCR program 100 while others are not affected or changed.

As those of skill in the art can further appreciate, conventional electronic healthcare record designs typically build a narrative using a system of menus. Such designs are limited in the ability to carry over usable data from previous visits, and therefore require the clinician to build an entirely new narrative from scratch for each visit. That is, typical electronic healthcare record programs lack narrative link-backs, since the data traditionally does not exist outside of the menu system that built the narrative. This results in very slow documentation and poor quality of resulting narratives. In addition, the data is no longer searchable and therefore does not provide usable data for reporting. According to aspects of the embodiments, in EHCR program 100 data from previous visits is carried over from visit to visit, and the subsequent narrative can either contain all of the previous narrative portion or have links directed to previous narratives. Users 120, 122, 124, 126 can therefore search through all of the narratives to find items/information of interest easily, and this can contribute to reduced costs and better healthcare for patient 118 according to aspects of the embodiments.

As those of skill in the art can further appreciate, typical electronic healthcare programs create separate templates for each type of visit and specialty. This results in poor data transfer between different visit types and specialties, resulting in poor communication between clinicians and low overall office efficiency. In addition, it multiplies the amount of system code that must be created and maintained by the product of specialties and the types of visits. Use of fewer templates, created as generically as possible, but with the addition of data fields in EHCR program 100 according to aspects of the embodiments obviates these deficiencies.

According to further aspects of the embodiments, because of the separation of layers 202-208, hot deployment of new software changes/patches/fixes can be more readily introduced with reduced probability of errors/crashes. In addition, EHCR program 100 can be fashioned to be installed in a “hot-deploy” manner, such that the code normally stored within platform layer 206 is stored in the run-time database; this results in a tightly coupled design that can be built and deployed together.

As those of skill in the art can now appreciate, and according to aspects of the embodiments, several improvements over prior art designs and technology result from use of separate layers 202-208. Such improvements include, but are not limited to (a) Separation of responsibilities between view layer 202, template layer 204, platform layer 206, and database layer 208 for improved and quicker data retrieval, processing, and rendering; (b) Separation of data input from automated narrative output; (c) Consolidation of all the template code across multiple medical specialties and multiple visit kinds into a single super-template and then selective display of only relevant data and input fields, at the view level; (d) Narrative link-backs to original data fields; and (e) Hot-deploy capability achieved by storing template code within the run-time database instead of storing the code inside platform layer 206.

As those of skill in the art can appreciate, existing technology does not allow multiple development efforts in parallel without impacting the core platform. This leads to longer development/test/release cycles, poor quality of resulting system, and fewer usable features for the end-user. In addition, each development/test/release cycle typically requires a reboot of the underlying platform, resulting in scheduled system downtime, or at least requiring a complex ordered restart of the entire server cluster. Aspects of the embodiments provide for a hot-deploy in the running system, which provides for substantially little or no downtime release of new versions of the software.

According to aspects of the embodiments, use of EHCR program 110 can substantially improve billing, compliance with federal and state laws, as well as modern medical practices, and can improve productivity. Because of the use of layers 202, 204, 206, 208, EHCR program 110 can be substantially continuously optimized for better network performance.

According to aspects of the embodiments, claims—the formal documents and submission thereof to government and private insurance companies—no longer carry over codes from previous claims, as each claim is self-supporting form codes that have been entered into their respective notes and other documentation sources. Such improvements to the claims are therefore better for compliance and makes it easier and intuitive to understand where the supporting claim information originates. According to aspects of the embodiments, the templates that are generated and stored within EHCR program 110 contain DX/CPT codes recommendations; upon completion of the notes, claims are automatically generated and processed. Billing and compliance are improved as the proper documents have been built into EHCR program 110 and noted in the narratives that are generated from notes.

According to further aspects of the embodiments, use of EHCR program 110 can provide one template for initial exam, re-exams, daily notes, and discharge; this makes the EHCR program 110 easier and more intuitive to use for users 120, 122, 124, 126. Such users can quickly become familiar with the template that is substantially used in the beginning of all experiences in a modern healthcare facility, and quickly learn the parts that particular pertain to their practices. Designing and implementing EHCR program 110 in this manner, according to aspects of the embodiments, substantially reduces information overload. The templates of EHCR program 110 can include color to make critical data stand out, and can use expandable and collapsible windows/interactive data entry fields to streamline the interfaces.

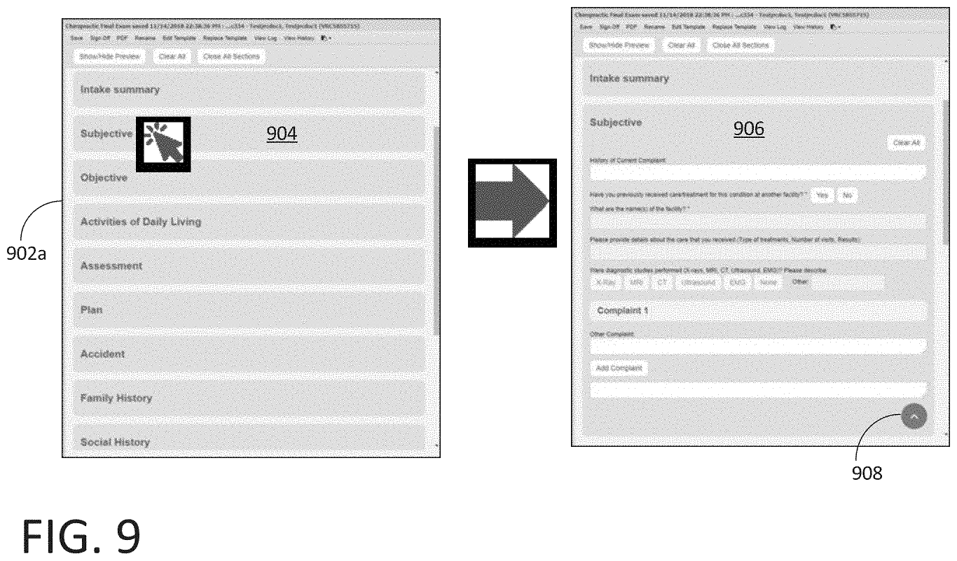

An example of such an expandable/collapsible interactive webpage/GUI windows is shown in FIG. 9. FIG. 9 illustrates a first and second view of GUI 902a,b, which is a template for use by a chiropractor. There is a “subjective” interactive data entry tab 904, which, when clicked in, opens/collapses between what is shown in GUI 902a and 902b; thus, upon clicking on “subjective interactive data entry tab 904a, “subjective” interactive data entry window 906 opens up and additional information can be input. When the user is done entering data/information, collapse button 908 can be clicked and window 906 collapses back to tab 904, and the view as shown in GUI 902a.

According to further aspects of the embodiments, use of the data entry windows/fields described above, and the overall design and implementation of EHCR program 110 means that less typing is needed, as values entered carry over between exams and daily notes as described above. Narratives, as discussed earlier, are automatically generated, and created in “prose-like” form to enhance readability, making the patient's data easier to understand and retain. Further as described above, there is access to external programs (See, e.g., Figure and the discussion thereof) that can be useful. Still further, according to aspects of the embodiments, navigation has become more intuitive due to the simplified, yet functional nature of the templates. Those of skill in the art can therefore now appreciate that all of these features save time and trouble to users 120-126.

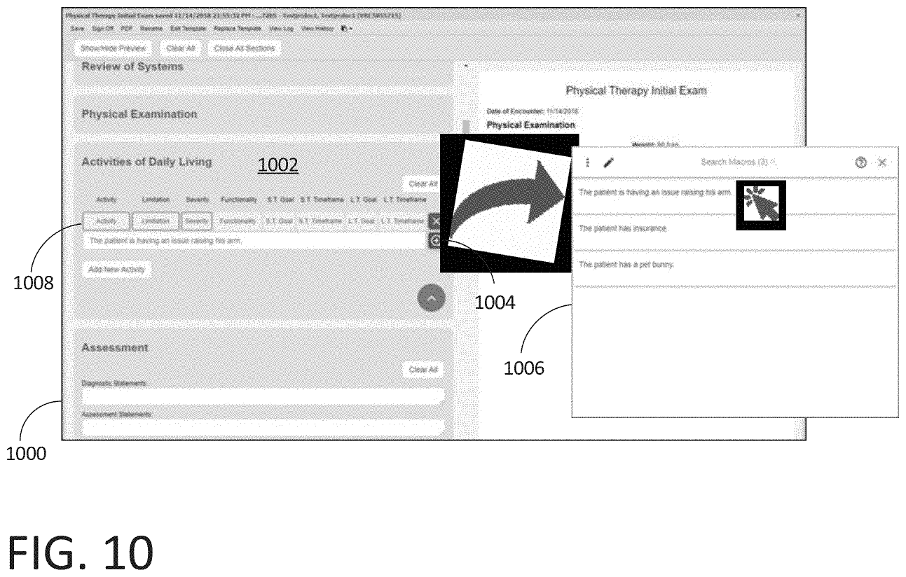

According to further aspects of the embodiments, the implementation and use of customizable macros in EHCR program 110 saves time for the users. A non-limiting example of such a macro field is shown in regard to FIG. 10. In FIG. 10, physical therapy GUI 1000 includes an activities of daily living record field 1002 and within field 1002 is activity button 1008. Clicking on activity button 1008 opens a window beneath it, in which a user can input additional data regard the PT's patient. Clicking on add button 1004 opens macro field 1006 into which the user can insert additional activities that could be pertinent to the assessment and treatment of the patient. For example, in this case the PT notes that the patient has trouble raising her arm, and that the patient has insurance and a pet bunny.

According to further aspects of the embodiments, the users 120-126 have access to search fields from the main templates to other relevant templates. Such templates are searchable by tags, can be sorted into ascending and descending lists, can be sorted by date and by template name, and other sort categories. In a substantially similar manner, such searching can also be performed, according to aspects of the embodiments, for previous visits to the respective medical healthcare facility; searching can be performed by date, appointment type, and/or patient name. According to further aspects of the embodiments, patient information pages contain a patient's personal information and history of visits, procedures, transactions, upcoming visit(s) schedule, and can further include a calendar view to make future appointments. Insurance information can be viewable on the patent information page, as well as summaries of submitted claims and their status. Additional important information that can be seen or readily accessible can include insurance plan information and agreements, images of a patient's clinical, diagnostic, and therapeutic procedures (such as x-ray images and the like).

According to further aspects of the embodiments, use of EHCR program 110 can facilitate better healthcare practices such as protecting personal information, such as “protected health information” (PHI). That is, EHCR program 110 can include safeguards and protections in full compliance with the latest legal updates and best practices in the industry, such that, for example, PHI data can be reviewed and wiped/stripped according to HIPAA restrictions.

According to further aspects of the embodiments, medical documents are available for viewing, including those that reference specific previous visits. Other features available for use with EHCR program 110 include a current tasks list, with corresponding due dates, priorities and status of the tasks. Notes that have been generated can be stored and made available for future use; such notes have an automatic “auto-save” capability built into each note as a default feature.

According to still further aspects of the embodiments, insurance care plans can be “built” that conform to respective requirements of various insurance plans. Prescription information, some of which is insurance plan dependent, can be integrated into the functionality of EHCR program 110 and an additional capability is the ability to create, fill, and forward order forms that fulfill the physician's orders. Other forms and features include referrals to different specialties.

According to aspects of the embodiments, various features are based on the layer-structure of EHCR program 110; these include: (a) polymorphism—the generation and use of a single interface that is viewable and usable by many different entities; (b) coordination and synchronization—intelligent sharing of relevant parts of notes across different specialties, the ability to allow multiple people to access and save information on the same document at the same time, and a multiple provider directory; and (c) reporting of outcome statistics, based on one or more patients.

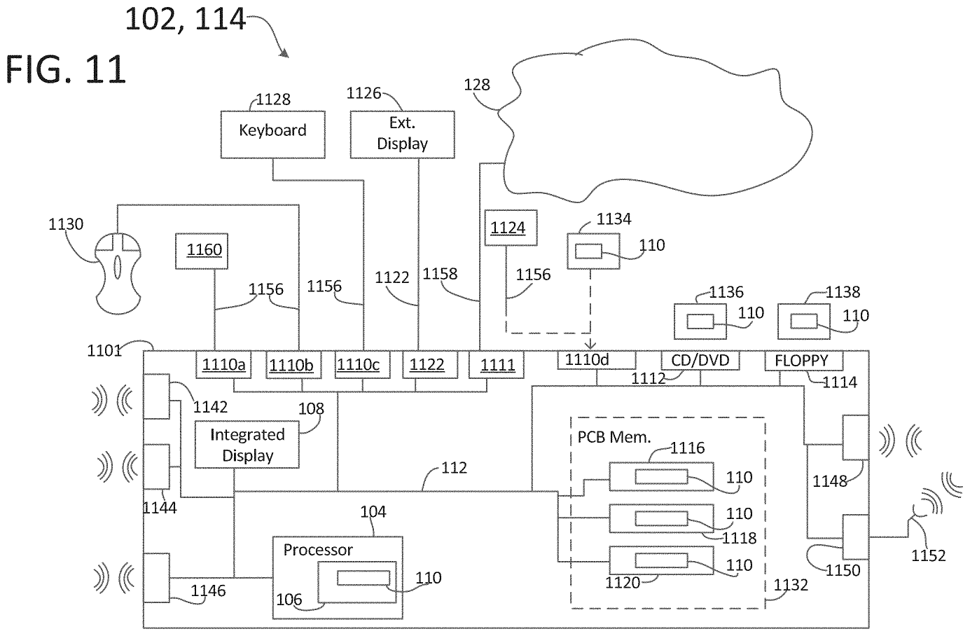

FIG. 11 illustrates a block diagram of PED/server 102/114 that can be used with EHCR program 110 according to aspects of the embodiments.

FIG. 11 illustrates a functional block diagram of PED/server 102/114 that can be used with EHCR program 110 according to aspects of the embodiments. As those of skill in the art can appreciate, many, if not most of the components shown and described in regard to FIG. 11 are equally applicable to both PED 102 and server 114, although each will have different, substantially non-germane capabilities. For the purposes of this discussion, the description and illustration in regard to FIG. 11 applies substantially equally to the aforementioned devices PED 102 and server 114, and therefore, in fulfilment of the dual purposes of clarity and brevity, reference has been made to only PED 102 in the subsequent discussion and not to both devices, although both—and other types of computing devices—can store and operate, or access and operate, EHCR program 110 according to aspects of the embodiments. Therefore, the discussion is only in reference to PED 102 and the detailed discussion of the other device(s) has been omitted from herein.

FIG. 11 illustrates a function block diagram of PED 102 as shown in FIG. 1. According to aspects of the embodiments, implementation of method 400 can occur in one or more of PED 102 and server 114, through use of EHCR program 110 according to aspects of the embodiments. Each of said devices comprises one or more processors 104, respectively, and memory 106, as well as data/command bus 112, and display 108, as described above.

Those of ordinary skill in the art in the field of the embodiments can appreciate that the functionality of the processor(s) 104 can be designed into various types of circuitry, including, but not limited to field programmable gate array structures (FPGAs), application specific integrated circuitry (ASICs), microprocessor based systems, among other types. A detailed discussion of the various types of physical circuit implementations does not substantively aid in an understanding of the embodiments, and as such has been omitted for the dual purposes of brevity and clarity. However, as well known to those of ordinary skill in the art, the systems and methods discussed herein can be implemented as discussed, and can further include programmable devices. Such programmable devices and/or other types of circuitry as previously discussed can include a processing unit, a system memory, and a system bus that couples various system components including the system memory to the processing unit. The system bus can be any of several types of bus structures including a memory bus or memory controller, a peripheral bus, and a local bus using any of a variety of bus architectures.

PED 102 includes, among other items, internal data/communications bus (bus) 112, processor(s) 104 (those of ordinary skill in the art can appreciate that in modern server systems, parallel processing is becoming increasingly prevalent, and whereas a single processor would have been used in the past to implement many or at least several functions, it is more common currently to have a single dedicated processor for certain functions (e.g., digital signal processors) and therefore could be several processors, acting in serial and/or parallel, as required by the specific application), universal serial bus (USB) port 1110, compact disk (CD)/digital video disk (DVD) read/write (R/W) drive 1112, floppy diskette drive 1114 (though less used currently, there are still many PCs that include this device), and memory 1132.

Memory 1132 (which can also be referred to as a data storage unit) itself can comprise hard disk drive (HDD) 1116 (these can include conventional magnetic storage media, but, as is becoming increasingly more prevalent, can include flash drive-type mass storage devices, among other types), ROM device(s) 1118 (these can include electrically erasable (EE) programmable ROM (EEPROM) devices, ultra-violet erasable PROM devices (UVPROMs), among other types), and random access memory (RAM) devices 1120. Usable with USB port 1110 is USB/FD/BT device 1134, and usable with CD/DVD R/W drive 1112 are CD/DVD disks 1136 (which can be both read and write-able). Usable with diskette drive 1114 are floppy diskettes 1138. Each of the memory storage devices, or the memory storage media (106, 1116, 1118, 1120, 1124, 1134, 1136, and 1138 among other types), can contain parts or components of, or in its entirety, executable software programming code (software; EHCR program 110 according to aspects of the embodiments that can implement part or all of the portions of the method described herein). Further, processor 104 itself can contain one or different types of memory storage devices (most probably, but not in a limiting manner, RAM memory storage media, that can store all or some of the components of EHCR program 110 according to aspects of the embodiments.

In addition to the above described components, PED 102 can also include external keyboard 1128, external display 1126 (while typically PED 102 can be a smart phone, or tablet, which would not typically have external devices connected to it, PED 102 can be a more conventional personal computer, laptop, or even tablet, all of which can or do have external components connected to them), and mouse 1130 (which can also be wireless, as can also external keyboard 1128, and external display 1126). All of these components are known to those of ordinary skill in the art, and this description includes all known and future variants of these types of devices. Internal display 108 can be any type of known display or presentation screen, such as liquid crystal displays (LCDs), light emitting diode displays (LEDs), plasma displays, cathode ray tubes (CRTs), among others. One or more user interface mechanisms can also be present such as a microphone, touch pad, touch screen, voice-recognition system, among other inter-active inter-communicative devices. Internal display 108 can also be a touch screen interface and thus can incorporate a touch-screen keyboard.

PED can access network/internet 128, either through a hard wired connection, via Ethernet interface 1111 directly, or wirelessly via one or more of BT transceiver/antenna 1144, near-field communication (NFC) transceiver/antenna 1146, and Wi-Fi/wireless transceiver antenna 1142. PED 102 can be coupled to other computing devices, via one or more networks. PED 102 can be part of a larger network configuration as in a global area network (GAN) (e.g., internet 128), which ultimately allows connection to various landlines.

PED 102 can be used to implement method 400 for operating an electronic healthcare record system (EHCR program 110) according to aspects of the embodiments. Hardware, firmware, software or a combination thereof may be used to perform the various steps and operations described herein. According to aspects of the embodiments, EHCR program 110 for carrying out the above discussed steps can be stored and distributed on multi-media storage devices such as devices 108, 1116, 1118, 1120, 1124, 1134, 1136, and 1138 (described above) or other form of media capable of portably storing information. These storage media may be inserted into, and read by, devices such as USB port 1110, disk drives 1112, 1114, among other types.

As also will be appreciated by one skilled in the art, the various functional aspects of the embodiments may be embodied in a wireless communication device, a telecommunication network, as a method, or in a computer program product. Accordingly, the embodiments may take the form of an entirely hardware embodiment or an embodiment combining hardware and software aspects. Further, the embodiments may take the form of a computer program product stored on a computer-readable storage medium having computer-readable instructions embodied in the medium. Any suitable computer-readable medium may be utilized, including hard disks, CD-ROMs, digital versatile discs (DVDs), optical storage devices, or magnetic storage devices such a floppy disk or magnetic tape. Other non-limiting examples of computer-readable media include flash-type memories or other known types of memories.

Further, those of ordinary skill in the art in the field of the embodiments can appreciate that such functionality can be designed into various types of circuitry, including, but not limited to field programmable gate array structures (FPGAs), application specific integrated circuitry (ASICs), microprocessor based systems, among other types. A detailed discussion of the various types of physical circuit implementations does not substantively aid in an understanding of the embodiments, and as such has been omitted for the dual purposes of brevity and clarity. However, as well known to those of ordinary skill in the art, the systems and methods discussed herein can be implemented as discussed and can further include programmable devices.

Such programmable devices and/or other types of circuitry as previously discussed can include a processing unit, a system memory, and a system bus that couples various system components including the system memory to the processing unit. The system bus can be any of several types of bus structures including a memory bus or memory controller, a peripheral bus, and a local bus using any of a variety of bus architectures. Furthermore, various types of computer readable media can be used to store programmable instructions. Computer readable media can be any available media that can be accessed by the processing unit. By way of example, and not limitation, computer readable media can comprise computer storage media and communication media. Computer storage media includes volatile and non-volatile as well as removable and non-removable media implemented in any method or technology for storage of information such as computer readable instructions, data structures, program modules or other data. Computer storage media includes, but is not limited to, RAM, ROM, EEPROM, flash memory or other memory technology, CDROM, digital versatile disks (DVD) or other optical disk storage, magnetic cassettes, magnetic tape, magnetic disk storage or other magnetic storage devices, or any other medium which can be used to store the desired information and which can be accessed by the processing unit. Communication media can embody computer readable instructions, data structures, program modules or other data in a modulated data signal such as a carrier wave or other transport mechanism and can include any suitable information delivery media.

The system memory can include computer storage media in the form of volatile and/or non-volatile memory such as read only memory (ROM) and/or random-access memory (RAM). A basic input/output system (BIOS), containing the basic routines that help to transfer information between elements connected to and between the processor, such as during start-up, can be stored in memory. The memory can also contain data and/or program modules that are immediately accessible to and/or presently being operated on by the processing unit. By way of non-limiting example, the memory can also include an operating system, application programs, other program modules, and program data.

The processor can also include other removable/non-removable and volatile/non-volatile computer storage media. For example, the processor can access a hard disk drive that reads from or writes to non-removable, non-volatile magnetic media, a magnetic disk drive that reads from or writes to a removable, non-volatile magnetic disk, and/or an optical disk drive that reads from or writes to a removable, non-volatile optical disk, such as a CD-ROM or other optical media. Other removable/non-removable, volatile/non-volatile computer storage media that can be used in the operating environment include, but are not limited to, magnetic tape cassettes, flash memory cards, digital versatile disks, digital video tape, solid state RAM, solid state ROM and the like. A hard disk drive can be connected to the system bus through a non-removable memory interface such as an interface, and a magnetic disk drive or optical disk drive can be connected to the system bus by a removable memory interface, such as an interface.

The embodiments discussed herein can also be embodied as computer-readable codes on a computer-readable medium. The computer-readable medium can include a computer-readable recording medium and a computer-readable transmission medium. The computer-readable recording medium is any data storage device that can store data which can be thereafter read by a computer system. Examples of the computer-readable recording medium include read-only memory (ROM), random-access memory (RAM), CD-ROMs and generally optical data storage devices, magnetic tapes, flash drives, and floppy disks. The computer-readable recording medium can also be distributed over network coupled computer systems so that the computer-readable code is stored and executed in a distributed fashion. The computer-readable transmission medium can transmit carrier waves or signals (e.g., wired or wireless data transmission through the Internet). Also, functional programs, codes, and code segments to, when implemented in suitable electronic hardware, accomplish or support exercising certain elements of the appended claims can be readily construed by programmers skilled in the art to which the embodiments pertains.

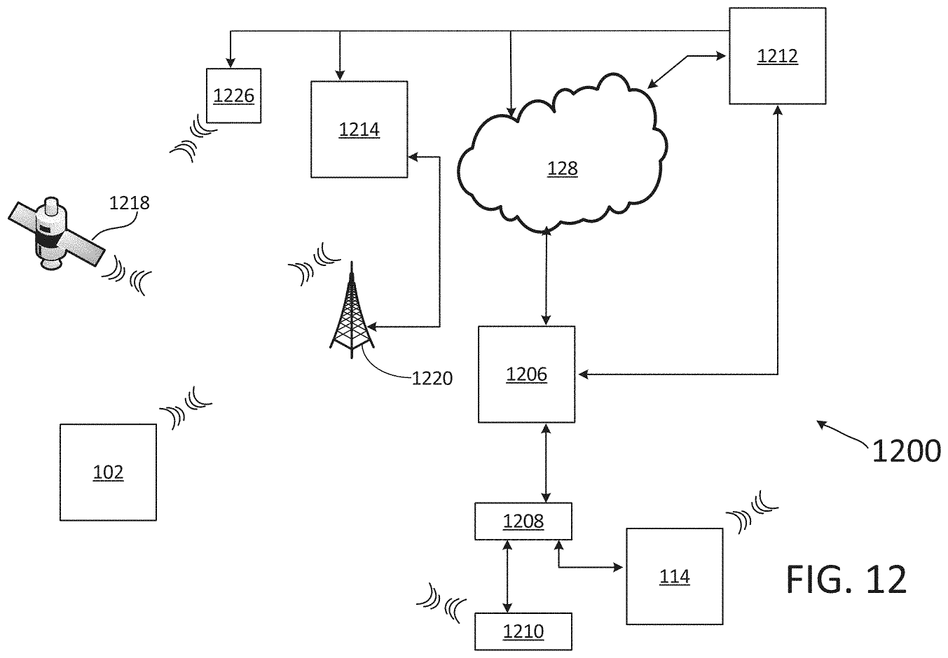

FIG. 12 illustrates a block diagram of network system (network) 1200 within which the system and method for operating an electronic healthcare record program can be implemented according to aspects of the embodiments. Much of the network system infrastructure shown in FIG. 12 is or should be known to those of skill in the art, so, in fulfillment of the dual purposes of clarity and brevity, a detailed discussion thereof shall be omitted. According to further aspects of the embodiments, system 100, described above in regard to FIG. 1, can be considered to be part of network 1200; thus, several of the components of system 100 are shown in network 1200.

According to aspects of the embodiments, users 118, 120, 122, 124, and 126 of the above described system and method can access and use EHCR program 110 on PED 102 and/or server 114; PED 102 can include, but are not limited to, so-called smart phones, tablets, personal digital assistants, notebook and laptop computers, and essentially any device that can access the internet and/or cellular phone service or can facilitate transfer of the same type of data in either a wired or wireless manner. For purposes of this discussion, as described above, users 118, 120, 122, 124, and 126 have been discussed as using only PED 102, i.e., a tablet (e.g., and iPad) though such discussion should be understood to be in a non-limiting manner in view of the discussion above about the other types of devices that can access, use, and provide such information.

In FIG. 12, users 118, 120, 122, 124, and 126 can access cellular service provider 1214, either through a wireless connection (cellular tower 1220) or via a communication system of an enterprise location (e.g., healthcare facility) that includes a wireless/wired interconnection (a “Wi-Fi” system that comprises, e.g., modulator/demodulator (modem) 1208, wireless router 1210, and server 114. Internet service provider (ISP) 1206 is the ISP of the healthcare care provider that accesses internet 128. Further, PED 102 can include near field communication (NFC), “Wi-Fi,” and Bluetooth (BT) communications capabilities as well, all of which are known to those of skill in the art and were described above in regard to FIG. 11.

According to further aspects of the embodiments, network 1200 further includes server 114 that can include wireless router 1210, and modem 1208. Thus, both PED 102 and server 114 can connect to ISP 1206 and internet 128 via internal modem 1208 to provide internet-based communications in the appropriate format to end users. Such communication pathways are well known and understand by those of skill in the art, and a further detailed discussion thereof is therefore unnecessary.

Either or both of PED 102 and server 114 can access communication satellites 1218 and their respective satellite communication systems control stations 1226 for near-universal communications capabilities, albeit at a much higher cost than convention “terrestrial” cellular services. PED 102 can also obtain positioning information when near or internal to a building (or arena/stadium) through the use of one or more of NFC/BT devices, the details of which are known to those of skill in the art. FIG. 12 also illustrates other components of network 1200 such as plain old telephone service (POTS) provider 1212.

According to further aspects of the embodiments, and as described above, network 1200 further comprises server 114 that includes EHCR program 110, wherein one or more processors, using known and understood technology, such as memory, data and instruction buses, and other electronic devices, can store and implement code that can implement the system, methods, and modes for operating an electronic healthcare records system according to aspects of the embodiments.

Aspects of the embodiments are directed towards systems, methods, and modes that performs electronic healthcare records data processing with systems that are not conventional in any manner. Aspects of the embodiments utilize a unique and non-obvious software structure that includes a plurality of layers that are functionally separate from each other, yet interact with each other, to create a synergistic effect in the manner of operating an electronic healthcare record system. Such synergistic effects include the ability to perform maintenance on one or more of the layers during run-time operations, and increasing the speed and efficiency of the electronic healthcare record system. Because the aspects of the embodiments utilize real devices such as PED 102, real data from real people stored in actual physical devices, and effectuate real-time processing on the stored data, the aspects of the embodiments cannot be said to be abstract; the aspects of the embodiments are directed to real, concrete improvements in the operation of electronic healthcare data keeping and recording systems. Such improvements as proffered by the aspects of the embodiments overcome the prior art difficulties as described above of slow operation, and the inability to make runtime changes to the code without disruptions.

The disclosed embodiments provide hardware, firmware, computer software, and a method for operating an electronic healthcare record system. It should be understood that this description is not intended to limit the embodiments. On the contrary, the embodiments are intended to cover alternatives, modifications, and equivalents, which are included in the spirit and scope of the embodiments as defined by the appended claims. Further, in the detailed description of the embodiments, numerous specific details are set forth to provide a comprehensive understanding of the claimed embodiments. However, one skilled in the art would understand that various embodiments may be practiced without such specific details.

Reference throughout the specification to “one embodiment” or “an embodiment” means that a particular feature, structure, or characteristic described in connection with an embodiment is included in at least one embodiment of the embodiments. Thus, the appearance of the phrases “in one embodiment” on “in an embodiment” in various places throughout the specification is not necessarily referring to the same embodiment. Further, the particular feature, structures, or characteristics may be combined in any suitable manner in one or more embodiments.

Although the features and elements of the embodiments are described in the embodiments in particular combinations, each feature or element can be used alone, without the other features and elements of the embodiments, or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

The above-described embodiments are intended to be illustrative in all respects, rather than restrictive, of the embodiments. Thus, the embodiments are capable of many variations in detailed implementation that can be derived from the description contained herein by a person skilled in the art. No element, act, or instruction used in the description of the present application should be construed as critical or essential to the embodiments unless explicitly described as such. Also, as used herein, the article “a” is intended to include one or more items.

All United States patents and applications, foreign patents, and publications discussed above are hereby incorporated herein by reference in their entireties.

1. An electronic healthcare record (EHCR) data processing method comprising:
running an EHCR program of code that comprises four separate but interactive layers of software code comprising:
a view layer of software code (view layer),
a template layer of software code (template layer),
a platform layer of software code (platform layer), and
a database layer of software code (database layer),
wherein the template layer is configured to:
store data entered as notes during visits by patients to a healthcare facility,
render the notes data in narrative form, and
obtain medical information from the database layer and generate treatment recommendations based on the obtained medical information.

2. The method according to claim 1, wherein the view layer is adapted to render template code to produce one or more interactive graphical user interfaces for entering and displaying data.

3. (canceled)

4. (canceled)

5. (canceled)

6. The method according to claim 1, wherein the template layer is further adapted to obtain data from both the platform layer and database layer.

7. The method according to claim 1, wherein the platform layer of code is adapted to respond to data requests from the template layer.

8. The method according to claim 7, wherein the platform layer, in response to data requests from the template layer, generates database search queries and retrieves the requested data from the appropriate database located in the database layer.

9. The method according to claim 1, wherein the database layer is adapted to store data in one or more separate database storage locations.

10. The method according to claim 9, wherein the database layer is further adapted to store graphical user interface (GUI) code.

11. The method according to claim 1, further comprising displaying the GUI code as a template GUI in a view layer of code.

12. A method for operating an electronic healthcare record system comprising:
operating software code that is organized into four separate layers, and wherein the software code comprises:
a view layer of software code (view layer),
a template layer of software code (template layer),
a platform layer of software code (platform layer), and
a database layer of software code (database layer);
rendering the template layer to display one or more interactive graphical user interfaces (GUIs) for use in entering, storing, retrieving, and processing electronic healthcare data, wherein the rendering occurs in the view layer;
maintaining data entered as notes documenting a visit by a patient, wherein the notes are maintained in the template layer and wherein a narrative is generated for each set of notes by software code maintained in the template layer;
requesting additional data through use of the one or more interactive GUIs;
obtaining requested data through the platform layer; and
storing requested data, notes data, and narratives in the database layer.

13. An electronic healthcare record (EHCR) data processing system comprising:
at least one electronic device, wherein the electronic device comprises:
at least one processor adapted to execute software code,
at least one display adapted to view results of executed software code, and
at least one memory storage adapted to store the executable software code; and
executable software code, wherein the executable software code comprises
a view layer of software code (view layer),
a template layer of software code (template layer),
a platform layer of software code (platform layer), and
a database layer of software code (database layer),
wherein the template layer is configured to:
store data entered as notes during visits by patients to a healthcare facility,
render the notes data in narrative form, and
obtain medical information from the database layer and generate treatment recommendations based on the obtained medical information.

14. The system according to claim 13, wherein the view layer is adapted to render template code to produce one or more interactive graphical user interfaces for entering and displaying data.

15. (canceled)

16. (canceled)

17. (canceled)

18. The system according to claim 13, wherein the template layer is further adapted to obtain data from both the platform layer and database layer.

19. The system according to claim 13, wherein the platform layer of code is adapted to respond to data requests from the template layer.

20. The system according to claim 19, wherein the platform layer, in response to data requests from the template layer, generates database search queries and retrieves the requested data from the appropriate database located in the database layer.

21. The system according to claim 13, wherein the database layer is adapted to store data in one or more separate database storage locations.

22. The system according to claim 21, wherein the database layer is further adapted to store graphical user interface (GUI) code.

23. The system according to claim 13, further comprising displaying the GUI code as a template GUI in a view layer of code.
